(19) United States
(12) Patent Application Publication
(10) Pub. No.: US 2023/0277066 A1
(43) Pub. Date: Sep. 7, 2023

(54) TEMPERATURE DETECTION METHOD AND APPARATUS, MEDIUM, AND ELECTRONIC DEVICE

(71) Applicant: ZHEJIANG UNIVIEW TECHNOLOGIES CO., LTD., Zhejang (CN)

(72) Inventor: Hui WANG, Zhejiang (CN)

(21) Appl. No.: 18/004,388

(22) PCT Filed: Dec. 24, 2020
(86) PCT No.: PCT/CN2020/139177

§ 371 Date: Jan. 5, 2023

(30) Foreign Application Priority Data
Jul. 6, 2020 (CN) 202010642012.X

(51) Int. Cl. A61B 5/01; A61B 5/00

(52) U.S. Cl. CPC A61B 5/01; A61B 5/0008; A61B 5/7221; A61B 5/7282

(57) ABSTRACT

A temperature detection method includes: acquiring real-time temperatures of a detected person, determining a temperature changing trend of the detected person to be a rising trend or a declining trend, and acquiring a real-time temperature changing speed; determining a higher limit of the real-time temperature changing speed and a lower limit of the real-time temperature changing speed; determining a temperature difference value corresponding to the higher limit according to the higher limit, and determining a temperature difference value corresponding to the lower limit according to the lower limit; determining a heat balance temperature higher limit according to the real-time temperatures, the temperature difference value corresponding to the higher limit, and the temperature changing trend, and determining a heat balance temperature lower limit according to the real-time temperatures, the lower limit, and the temperature changing trend; and determining a temperature detection result.

This application claims the priority of Chinese Patent Application No. 202010642012. X, filed with the Chinese Intellectual Property Administration on Jul. 6, 2020, the disclosure of which is incorporated herein by reference in its entirety.

### TECHNICAL FIELD

The present application relates to the technical field of temperature detection, for example, to a temperature detection method, a temperature detection apparatus, a medium and an electronic device.

### BACKGROUND

With the rapid development of science and technology, people pay more and more attention to safety of human body. In some scenarios, it is very important to perform non-contact temperature measurement on the human body.

The non-contact human body temperature measurement method uses a forehead thermometer for human body temperature measurement. In the outdoor scene, since the body surface temperature of the human body fluctuates greatly due to the influence of the environment, it takes a certain period of time to restore the normal body surface temperature, which leads to the temperature measurement results obtained by the forehead thermometer often not being able to truly reflect temperature of the human body. Due to the limitations of some scenarios, such as the entrances and exits of stations, shopping malls, and office buildings, it is not possible to test everyone for a sufficient long time, and the accuracy of the temperature test results cannot be guaranteed.

### SUMMARY

A temperature detection method, a temperature detection apparatus, a medium and an electronic device are provided according to the present application, which may calculate heat balance temperature values by performing a temperature trend detection, and determine a human body temperature detection result according to relationships between an alarm temperature threshold value and the heat balance temperature values to achieve the object of improving the accuracy of human body temperature detection.

A temperature detection method is provided, the method includes the following.

Real-time temperatures of a detected person is acquired, a temperature changing trend of the detected person is determined to be a rising trend or a declining trend according to the real-time temperatures, and a real-time temperature changing speed is acquired according to the real-time temperatures.

A higher limit of the real-time temperature changing speed and a lower limit of the real-time temperature changing speed are determined according to the real-time temperature changing speed.

A temperature difference value corresponding to the higher limit of the real-time temperature changing speed is determined according to the higher limit of the real-time temperature changing speed, and a temperature difference value corresponding to the lower limit of the real-time temperature changing speed is determined according to the lower limit of the real-time temperature changing speed.

A heat balance temperature higher limit is determined according to the real-time temperatures, the temperature difference value corresponding to the higher limit of the real-time temperature changing speed, and the temperature changing trend. A heat balance temperature lower limit is determined according to the real-time temperatures, the lower limit of the real-time temperature changing speed, and the temperature changing trend.

A temperature detection result of the detected person is determined according to a relationship between the heat balance temperature higher limit and an alarm temperature threshold and a relationship between the heat balance temperature lower limit and the alarm temperature threshold.

A temperature detection apparatus is further provided, which includes: a real-time temperature changing speed acquisition module, a limit value determination module, a temperature difference value determination module, a heat balance temperature range determination module and a detection result determination module.

The real-time temperature changing speed acquisition module is configured to acquire real-time temperatures of a detected person, determine a temperature changing trend of the detected person to be a rising trend or a declining trend according to the real-time temperatures, and acquire a real-time temperature changing speed according to the real-time temperatures.

The limit value determination module is configured to determine a higher limit of the real-time temperature changing speed and a lower limit of the real-time temperature changing speed according to the real-time temperature changing speed.

The temperature difference value determination module is configured to determine a temperature difference value corresponding to the higher limit of the real-time temperature changing speed according to the higher limit of the real-time temperature changing speed, and determine a temperature difference value corresponding to the lower limit of the real-time temperature changing speed according to the lower limit of the real-time temperature changing speed.

The heat balance temperature range determination module is configured to determine a heat balance temperature higher limit according to the real-time temperatures, the temperature difference value corresponding to the higher limit of the real-time temperature changing speed, and the temperature changing trend, and determine a heat balance temperature lower limit according to the real-time temperatures, the lower limit of the real-time temperature changing speed, and the temperature changing trend.

The detection result determination module is configured to determine a temperature detection result of the detected person according to a relationship between the heat balance temperature higher limit and an alarm temperature threshold and a relationship between the heat balance temperature lower limit and the alarm temperature threshold.

A computer-readable storage medium is further provided, which stores a computer program, and when the computer program is executed by a processor, the temperature detection method as described in the embodiments of the present application is implemented.

An electronic device is further provided, which includes a memory, a processor, and a computer program stored on the memory and operable on the processor, and the processor, when executing the computer program, implements the temperature detection method as described in the embodiments of the present application.

### BRIEF DESCRIPTION OF DRAWINGS

FIG. 1 is a flow chart of a temperature detection method according to an embodiment of the present application;

FIG. 2 is a schematic diagram of a temperature trend detection window according to an embodiment of the present application;

FIG. 3 is a schematic diagram of a temperature trend detection window according to an embodiment of the present application;

FIG. 4 is a schematic structural diagram of a temperature detection apparatus according to an embodiment of the present application;

FIG. 5 is a schematic structural diagram of an electronic device according to an embodiment of the present application.

### DETAILED DESCRIPTION

The present application is described hereinafter in conjunction with the drawings and embodiments. The embodiments described here are merely used to explain the present application rather than limiting the present application. For ease of description, only some structures rather than all structures related to the present application are shown in the drawings.

Some exemplary embodiments are described as processes or methods depicted as flowcharts. Although the flowcharts describe steps as being sequential performed, many of the steps may be performed in parallel, concurrently, or simultaneously. Additionally, the order of various steps may be rearranged. The process may be terminated when its operations are completed, but may also have additional steps not included in the drawings. The processing may correspond to a method, function, procedure, subroutine, subprogram, or the like.

There is a corresponding relationship between a body surface temperature and a body temperature of a human body, based on the corresponding relationship, the corresponding human body temperature may be calculated by measuring the body surface temperature. The forehead thermometers and ear thermometers sold on the market are all based on this principle.

The body surface temperature of the human body will change with changes in factors such as ambient temperature, air humidity, ambient wind force, body surface clothing coverage of human body, and movement status of human body. Therefore, when the above factors change, the phenomenon of inaccurate temperature measurement may occur.

For example, when a person walks from a high-temperature indoor environment to a relatively low-temperature gate for temperature measurement, the body surface temperature of the human body may be relatively high, and when a person walks from a low-temperature outdoor environment to an indoor environment for temperature measurement, the body surface temperature of the human body may be relatively low. The body surface temperatures of the human body in these cases are in a state of non-heat balance, and measuring the temperature of the body surface at these times, relatively inaccurate temperature values may be obtained.

The body surface temperature of the human body is affected by heat production in the body and heat dissipation through the body surface. The heat production methods of the human body include basal metabolic heat production, heat production by exercise, heat production by human vaso contraction and relaxation, and heat production by human body tremor, etc. The human body heat dissipation methods include heat dissipation by conduction, heat dissipation by convection, heat dissipation by radiation, and heat dissipation by evaporation. When the above-mentioned factors affecting heat production and heat dissipation appear, they will all lead to changes in the body surface temperature of the human body. When the heat produced in the body is more than the heat dissipated from the body surface, the body surface temperature rises. When the heat produced in the body is less than the heat dissipated from the body surface, the body surface temperature will decline. When the heat produced in the human body is equal to the heat dissipated from the body surface, the body surface temperature will maintain a relatively constant value. At this time, the body surface temperature may represent the body temperature relatively accurately.

In order to better measure the temperature of the human body, it is necessary to implement different temperature measurement strategies according to the changing trend of the body surface temperature to ensure the accuracy of the measurement results.

In the practical temperature measurement application, the temperature measurement speed is also an important indicator, which will affect the practical use experience. For example, when the temperature measurement is clocked in during the peak commute, the low temperature measurement speed will cause the queue to be too long, which, in one aspect, will affect the speed of getting on and off work, and in another aspect, will lead to gathering of a large number of people and increase the risk of cross-infection.

Therefore, it is necessary to integrate the body temperature changing trend and the temperature measurement speed to obtain a temperature measurement solution that can take both aspects into consideration. In this solution, the body temperature detection is to predict the body temperature by detecting the body surface temperature, and all the temperatures described in the following process are the body surface temperature.

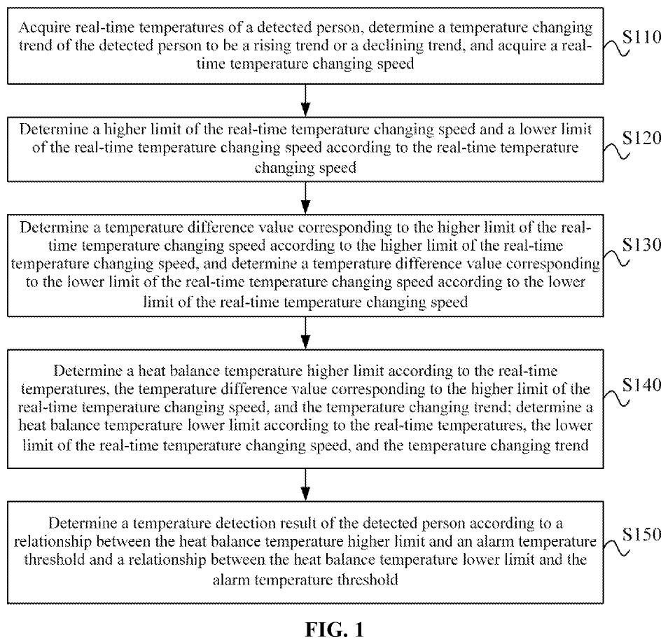

FIG. 1 is a flow chart of a temperature detection method according to an embodiment of the present application. This embodiment may be applied to the situation of body temperature detection. This method may be executed by a temperature detection apparatus according to an embodiment of the present application. The temperature detection apparatus may be implemented by means of software and/or hardware, and may be integrated into electronic devices with corresponding computation capabilities.

As shown in FIG. 1, the temperature detection method includes: S110, S120, S130, S140 and S150.

In S110, real-time temperatures of a detected person is acquired, a temperature changing trend of the detected person is determined to be a rising trend or a declining trend, and a real-time temperature changing speed is acquired.

The detected person may be a staff, a student and a traveler. The real-time temperature of the detected person may be obtained through a temperature gun, etc. The real-time temperature of the detected person may be one temperature value or multiple temperature values, for example, if the body temperature gun is controlled to collect body temperatures according to a preset frequency, multiple body temperature values may be obtained, for example, if temperature values are collected once every 0.2 seconds, then in a two-second acquisition time, ten real-time temperature values may be obtained.

After the temperature values of multiple real-time temperatures are collected, the temperature changing trend of the detected person may be determined according to the change rules of the temperature values of the multiple real-time temperatures. The temperature changing trend may include a rising trend, a declining trend, and a flat trend. If the body temperature of the detected person is on a flat trend, it means that the measured real-time temperature are consistent with the actual heat balance temperature of the detected person, and a determination can be made directly.

The real-time temperature changing speed may be determined according to one or more slopes between multiple temperature values, for example, may be determined according to an average value of slopes between two adjacent values of the multiple temperature values. For example, a slope formed by a temperature value 1 and a temperature value 2, a slope formed by the temperature value 2 and a temperature value 3, and so on, and then all obtained slopes are averaged to obtain the real-time temperature changing speed.

In this solution, since the multiple temperature values collected are analyzed to determine the temperature changing rule, there may also be a case where multiple temperature values are unstable, for example, the rising trends and the falling trends appear alternately. In this case, a reminder can be given to inform the staff to re-detect the temperature of the detected person or to check the connection and working conditions of the device to eliminate the errors caused by the device and the errors caused by human manipulation of the physical objects during the collection process.

In this embodiment, optionally, the acquiring real-time temperatures of a detected person; and determining a temperature changing trend of the detected person to be a rising trend or a declining trend includes the following.

The real-time temperatures of the detected person are sampled according to a preset period; an average slope of real-time temperature sampled points of the detected person is determined through a preset temperature trend detection window; and if the average slope is greater than 0, it is determined that the temperature changing trend is the rising trend; if the average slope is less than 0, it is determined that the temperature changing trend is the declining trend.

The preset period may be 0.2 seconds, 0.5 seconds, or a longer or shorter time. After sampling, the temperature sampled points may be displayed in the temperature trend detection window. Specifically, the temperature trend detection window may be provided in the temperature gun, and may also be provided in an intelligent terminal, such as an intelligent mobile terminal, a computer, and a tablet computer, connected in a communication manner to the temperature gun.

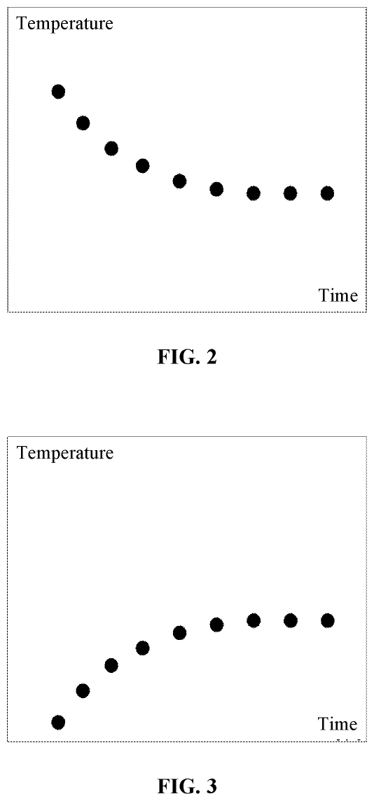

FIG. 2 and FIG. 3 are schematic diagrams of the temperature trend detection window according to an embodiment of the present application. As shown in FIG. 2 and FIG. 3, if the temperature gradually increases over time, the average slope is greater than 0, it indicates that the temperature changing trend of the body surface temperature of the detected person is the rising trend, and if the average slope is less than 0, then it indicates that the temperature changing trend of the body surface temperature of the detected person is the declining trend.

On the basis of the above technical solution, optionally, the method further includes the following.

If the temperature changing trend is the rising trend, then a temperature increasing speed in the temperature trend detection window is calculated and used as the real-time temperature changing speed; and if the temperature changing trend is the declining trend, a temperature decreasing speed in the temperature trend detection window is calculated and used as the real-time temperature changing speed.

In the rising trend and the declining trend, the temperature increasing speed or temperature decreasing speed in the temperature trend detection window may be calculated as the real-time temperature changing speed.

For example, if the temperature is on a rising trend, an average temperature increasing speed SpeedTempInc in the temperature trend detection window is calculated.

If the temperature is on a declining trend, an average temperature decreasing speed SpeedTempDec in the temperature trend detection window is calculated.

In S120, a higher limit of the real-time temperature changing speed and a lower limit of the real-time temperature changing speed are determined according to the real-time temperature changing speed.

The higher limit of the real-time temperature changing speed and the lower limit of the real-time temperature changing speed may be determined according to the real-time temperature changing speed and a preset empirical coefficient.

Optionally, the real-time temperature changing speed includes a temperature increasing speed and a temperature decreasing speed.

The determining a higher limit of the real-time temperature changing speed and a lower limit of the real-time temperature changing speed according to the real-time temperature changing speed includes the following.

The higher limit of the real-time temperature changing speed is determined according to the products of the temperature increasing speed and a preset higher limit coefficient; and the lower limit of the real-time temperature changing speed is determined according to the products of the temperature decreasing speeds and a preset lower limit coefficient.

In this embodiment, optionally, the determining a higher limit of the real-time temperature changing speed and a lower limit of the real-time temperature changing speed according to the real-time temperature changing speed includes the following.

The higher limit of the real-time temperature changing speed is determined according to the products of the real-time temperature changing speed and a preset higher limit coefficient; and the lower limit of the real-time temperature changing speed is determined according to the products of the real-time temperature changing speed and a preset lower limit coefficient.

The absolute value SpeedTemp of the temperature changing speed SpeedTempInc or SpeedTempDec may be taken to calculate the higher limit of the real-time temperature changing speed SpeedTempLow and the lower limit of the real-time temperature changing speed SpeedTempHig:
SpeedTempLow=SpeedTemp*KLow;
SpeedTempHig=SpeedTemp*KHig;
The KLow is an empirical coefficient less than 1, and KHig is an empirical coefficient greater than 1.

Controlling the temperature changing speed through the empirical coefficient can achieve the effect that the heat balance temperature is calculated through a range value, which can ensure that the finally analyzed heat balance temperature is more accurate.

In S130, a temperature difference value corresponding to the higher limit of the real-time temperature changing speed is determined according to the higher limit of the real-time temperature changing speed, and a temperature difference value corresponding to the lower limit of the real-time temperature changing speed is determined according to the lower limit of the real-time temperature changing speed.

The temperature difference value may be the difference between a heat balance temperature and a real-time temperature. The temperature difference value corresponding to the higher limit of the real-time temperature changing speed and the temperature difference value corresponding to lower limit of the real-time temperature changing speed may be determined according to the higher limit of the real-time temperature changing speed, the lower limit of the real-time temperature changing speed and the changing speed low limit.

In this technical solution, the characteristic that when the temperature difference value is larger, the changing speed is higher can be utilized to construct a table of the mapping relationship between the magnitudes of the changing speed and the temperature difference values. The respective temperature difference values corresponding to an obtained higher limit of the real-time temperature changing speed and an obtained lower limit of the real-time temperature changing speed may be determined by looking up from the table according to the obtained higher limit of the real-time temperature changing speed and the obtained lower limit of the real-time temperature changing speed.

The lower limit of the real-time temperature changing speed SpeedTempLow and the higher limit of the real-time temperature changing speed SpeedTempHig may be used to obtain temperature difference values TempDiffCurLow and TempDiffCurHigh at the current speed respectively by looking up a table using the linear interpolation method.

In S140, a heat balance temperature higher limit is determined according to the real-time temperatures, the temperature difference value corresponding to the higher limit of the real-time temperature changing speed, and the temperature changing trend; a heat balance temperature lower limit is determined according to the real-time temperatures, the lower limit of the real-time temperature changing speed, and the temperature changing trend.

In this solution, the heat balance temperature may be determined according to the temperature changing trend, the currently measured real-time temperature, the temperature difference value corresponding to the higher limit of the real-time temperature changing speed determined above and the temperature difference value corresponding to the lower limit of the real-time temperature changing speed determined above.

Optionally, the determining a heat balance temperature higher limit and a heat balance temperature lower limit according to the real-time temperatures and the temperature difference value corresponding to the higher limit of the real-time temperature changing speed and the temperature difference value corresponding to the lower limit of the real-time temperature changing speed, and according to the temperature changing trend includes the following.

When the temperature changing trend is the rising trend, the heat balance temperature higher limit is the sum of the real-time temperature and the temperature difference value corresponding to the higher limit of the real-time temperature changing speed; and the heat balance temperature lower limit is the sum of the real-time temperature and the temperature difference value corresponding to the lower limit of the real-time temperature changing speed.

When the temperature changing trend is the declining trend, the heat balance temperature higher limit is a difference between the real-time temperature and the temperature difference value corresponding to the lower limit of the real-time temperature changing speed; and the heat balance temperature lower limit is a difference between the real-time temperature and the temperature difference value corresponding to the higher limit of the real-time temperature changing speed.

According to TempDiffCurLow, TempDiffCurHigh, and the current temperature TempCur, the temperature values under Heat balance TempHeatBalanceLow and TempHeatBalanceHig are calculated.

If the temperature is on a rising trend, then
TempHeatBalanceLow=TempCur+TempDiffCurLow; and
TempHeatBalanceHig=TempCur+TempDiffCurHig.

If the temperature is on a declining trend, then
TempHeatBalanceLow=TempCur−TempDiffCurHig; and
TempHeatBalanceHig=TempCur−TempDiffCurLow.

Through the above calculations, the heat balance temperature higher limit and the heat balance temperature lower limit may be obtained respectively for different temperature changing trends.

In S150, a temperature detection result of the detected person is determined according to a relationship between the heat balance temperature higher limit and an alarm temperature threshold and a relationship between the heat balance temperature lower limit and the alarm temperature threshold.

The body temperature threshold for human body fever alarm is generally 37.3 degrees, but the corresponding body surface temperatures under different ambient temperatures are different, therefore, it is necessary to establish a table of mapping relationship of ambient temperatures to alarm body surface temperatures.

The table of mapping relationship of the ambient temperatures to the alarm body surface temperatures may be established through experiments.

According to the current ambient temperature TempEnv, the alarm temperature threshold ThrTempAlm under the current ambient temperature is acquired by looking up the table through the linear interpolation method. For example, if the ambient temperature is 20 degrees, the alarm body surface temperature threshold may be lower than 37.3 degrees, such as 37 degrees.

Here, it may be divided into two cases, namely a rising trend and a declining trend.

The object of this embodiment is to detect the person whose heat balance temperature exceeds the alarm temperature threshold, that is, to detect the person with high fever. Therefore, on the rising trend, the heat balance temperature lower limit corresponding to the lower limit of the real-time temperature changing speed may be determined. If the heat balance temperature lower limit exceeds the alarm temperature threshold, it is determined that the detected person is in a state of high fever. In the declining stage, the heat balance temperature lower limit corresponding to the higher limit of the real-time temperature changing speed may be determined, and if the heat balance temperature lower limit exceeds the alarm temperature threshold, it is determined that the detected person is in a state of high fever.

In this embodiment, the determining a temperature detection result of the detected person according to a relationship between the heat balance temperature higher limit and an alarm temperature threshold and a relationship between the heat balance temperature lower limit and the alarm temperature threshold, includes the following.

When the temperature changing trend is the rising trend, if the alarm temperature threshold is less than the heat balance temperature lower limit, it is determined that the temperature detection result is an abnormal high temperature; and if the alarm temperature threshold is greater than the heat balance temperature higher limit, it is determined that the temperature detection result is a normal body temperature.

When the temperature changing trend is the declining trend, if the alarm temperature threshold is less than the heat balance temperature lower limit, it is determined that the temperature detection result is an abnormal high temperature; and if the alarm temperature threshold is greater than the heat balance temperature higher limit, it is determined that the temperature detection result is a normal body temperature.

With such a setting, it may be determined whether the detected person is in a state of high fever according to the magnitude relationship between the alarm temperature threshold and the heat balance temperature limits.

In the technical solution according to an embodiment of the present application, the real-time temperatures of the detected person are acquired; and it is determined that the temperature changing trend of the detected person is a rising trend or a declining trend, and the real-time temperature changing speed is acquired; the higher limit of the real-time temperature changing speed and the lower limit of the real-time temperature changing speed are determined according to the real-time temperature changing speed; the temperature difference value corresponding to the higher limit of the real-time temperature changing speed and the temperature difference value corresponding to the lower limit of the real-time temperature changing speed are determined respectively according to the higher limit of the real-time temperature changing speed and the lower limit of the real-time temperature changing speed; the heat balance temperature higher limit and the heat balance temperature lower limit are determined according to the real-time temperatures and the temperature difference value corresponding to the higher limit of the real-time temperature changing speed and the temperature difference value corresponding to the lower limit of the real-time temperature changing speed; and the temperature detection result of the detected person is determined according to the relationship between the heat balance temperature higher limit and the alarm temperature threshold and the relationship between the heat balance temperature lower limit and the alarm temperature threshold. By adopting the technical solution according to this application, the heat balance temperature value may be calculated by performing temperature trend detection, and the human body temperature detection result can be determined according to the relationships between the alarm temperature threshold and the heat balance temperature values, so as to achieve the object of improving the accuracy of human body temperature detection.

On the basis of the above technical solution, optionally, before the determining the temperature detection result of the detected person according to the relationship between the heat balance temperature higher limit and an alarm temperature threshold and the relationship between the heat balance temperature lower limit and the alarm temperature threshold, the method further includes the following.

A balancing time higher limit is determined according to the higher limit of the real-time temperature changing speed and a balancing time lower limit is determined according to the lower limit of the real-time temperature changing speed.

Specifically, the balancing time higher limit and the balancing time lower limit are the parameters in the table of mapping relationship between the magnitudes of changing speed and the temperature difference values, that is, the time value required for the temperature to reach balance, and according to the time value required for the temperature to reach balance and through determination, the temperature measurement speed is ensured.

Specifically, the table of mapping relationship between the magnitudes of changing speed and the temperature difference values, the table items may be as follows.
Table item 1: temperature difference 1, temperature changing speed 1, time for temperature reaching balance 1;
Table item 2: temperature difference 2, temperature changing speed 2, time for temperature reaching balance 2;
Table item N: temperature difference N, temperature changing speed N, time for temperature reaching balance N.

The lower limit of the real-time temperature changing speed SpeedTempLow and the higher limit of the real-time temperature changing speed SpeedTempHig are used respectively to obtain temperature difference values at the current speed TempDiffCurLow and TempDiffCurHigh and balancing time values TimeHeatBalanceLow and TimeHeatBalanceHig by looking up a table using the linear interpolation method.

The temperature values under heat balance TempHeatBalanceLow and TempHeatBalanceHig are calculated according to TempDiffCurLow, TempDiffCurHigh, and current temperature TempCur.

If the temperature is on a rising trend, then TempHeatBalanceLow (temperature value under heat balance)=TempCur+TempDiffCurLow (temperature difference value under current speed); and TempHeatBalanceHig (temperature value under heat balance)=TempCur+TempDiffCurHig (temperature difference value under current speed).

If the temperature is in a declining trend, then TempHeatBalanceLow=TempCur−TempDiffCurHig; and TempHeatBalanceHig=TempCur−TempDiffCurLow.

Correspondingly, the determining a temperature detection result of the detected person according to a relationship between the heat balance temperature higher limit and an alarm temperature threshold and a relationship between the heat balance temperature lower limit and the alarm temperature threshold, includes the following.

When the temperature changing trend is the rising trend, if the alarm temperature threshold is greater than the heat balance temperature lower limit and less than the heat balance temperature higher limit, then a relationship between a balancing time for the real-time temperature to rise to the alarm temperature threshold and an acceptable measurement time threshold is determined; and if a balancing time for the real-time temperature to rise to the alarm temperature threshold is less than or equal to the acceptable measurement time threshold, then temperature measurement is continued; and if a balancing time for the real-time temperature to rise to the alarm temperature threshold is greater than the acceptable measurement time threshold, issuing a warning message of temperature instability.

When the temperature changing trend is the declining trend, if the alarm temperature threshold is greater than the heat balance temperature lower limit and less than the heat balance temperature higher limit, then determining a relationship between a balancing time for the real-time temperature to decline to the alarm temperature threshold and the acceptable measurement time threshold; if the time for the real-time temperature to decline to the alarm temperature threshold is less than or equal to the acceptable measurement time threshold, then the temperature measurement is continued; and if the time for the real-time temperature to decline to the alarm temperature threshold is greater than the acceptable measurement time threshold, a warning message of temperature instability is issued.

The acceptable measurement time threshold may be determined according to the detection environment, for example, in a train station, due to the large number of passengers, it can often be set short, for example, 10 seconds; and if in an office, due to less cases of coming in and out of workers, it may be set long, for example, 25 seconds.

Further since the balancing times corresponding to the rising state and the declining state respectively have been determined previously, it may be determined whether to wait for the length of the balancing time before performing body temperature detection on the detected person according to the comparison of the balancing time with the measurement time threshold.

If it is on the rising trend, and the alarm temperature threshold is greater than the heat balance temperature lower limit and less than the heat balance temperature higher limit, then the relationship between the balancing time for rising from the real-time temperature to the alarm temperature threshold and the acceptable measurement time threshold is determined.

If the balancing time for rising from the real-time temperature to the alarm temperature threshold is less than or equal to the acceptable measurement time threshold, then the temperature measurement is continued;

If the balancing time for rising from the real-time temperature to the alarm temperature threshold is greater than the acceptable measurement time threshold, a warning message of temperature instability is issued.

Through such a setting, different treatments can be carried out for the detected person in different situations, so as to improve the accuracy of body temperature detection and the feasibility of the solution, fully consider the time efficiency of body temperature detection, and better deploy for temperature detections in various environments.

When the body temperature is on a rising trend, if the temperature is greater than the temperature alarm threshold ThrTempAlm, a high temperature alarm is warned; if the temperature is lower than the temperature alarm threshold ThrTempAlm, and it is determined if the alarm temperature threshold ThrTempAlm is less than the TempHeatBalanceLow, then a high temperature alarm is directly outputted; if the alarm temperature threshold ThrTempAlm is greater than the TempHeatBalanceHig, then a normal temperature measurement result is outputted directly; if the alarm temperature threshold ThrTempAlm is between the TempHeatBalanceLow and the TempHeatBalanceHig, and according to the time required for the temperature to reach balance TimeHeatBalanceHig when the temperature is the TempHeatBalanceHig, and according to a uniform deceleration formula, the maximum time TimeAlarmHeatBalanceMax required for the temperature to reach the alarm temperature threshold ThrTempAlm is calculated, and it is determined if the TimeAlarmHeatBalanceMax is less than or equal to the acceptable measurement time threshold ThrTimeWait, then the continuous temperature measurement state is maintained; and if the TimeAlarmHeatBalanceMax is greater than the acceptable measurement time threshold ThrTimeWait, then temperature instability is warned, and measurement is performed later.

When the body temperature is on a declining trend, if the temperature is lower than the temperature alarm threshold ThrTempAlm, a normal temperature measurement result is outputted directly; if the temperature is greater than the temperature alarm threshold ThrTempAlm, and it is determined if the alarm temperature threshold ThrTempAlm is less than the TempHeatBalanceLow, then a high temperature alarm is directly outputted; if the alarm temperature threshold ThrTempAlm is greater than the TempHeatBalanceHig, then the normal temperature measurement result is outputted directly; if the alarm temperature threshold ThrTempAlm is between the TempHeatBalanceLow and the TempHeatBalanceHig, and according to the time required for the temperature to reach balance TimeHeatBalanceLow when the temperature is the TempHeatBalanceLow, and according to a uniform deceleration formula, the time TimeAlarmHeatBalanceMax required for the temperature to reach the alarm temperature threshold ThrTempAlm is calculated, and it is determined if the TimeAlarmHeatBalanceMax is less than or equal to the acceptable measurement time threshold ThrTimeWait, then the continuous temperature measurement state is maintained; and if the TimeAlarmHeatBalanceMax is greater than the acceptable measurement time threshold ThrTimeWait, then temperature instability is warned, and measurement is performed later.

FIG. 4 is a schematic structural diagram of a temperature detection apparatus according to an embodiment of the present application. As shown in FIG. 4, the temperature detection apparatus includes: a real-time temperature changing speed acquisition module 410, a limit value determination module 420, a temperature difference value determination module 430, a heat balance temperature range determination module 440, and a detection result determination module 450.

The real-time temperature changing speed acquisition module 410 is configured to acquire real-time temperatures of a detected person, determine a temperature changing trend of the detected person to be a rising trend or a declining trend according to the real-time temperatures, and acquire a real-time temperature changing speed according to the real-time temperatures.

The limit value determination module 420 is configured to determine a higher limit of the real-time temperature changing speed and a lower limit of the real-time temperature changing speed according to the real-time temperature changing speed.

The temperature difference value determination module 430 is configured to determine a temperature difference value corresponding to the higher limit of the real-time temperature changing speed according to the higher limit of the real-time temperature changing speed and determine a temperature difference value corresponding to the lower limit of the real-time temperature changing speed according to the lower limit of the real-time temperature changing speed.

The heat balance temperature range determination module 440 is configured to determine a heat balance temperature higher limit according to the real-time temperatures, the temperature difference value corresponding to the higher limit of the real-time temperature changing speed, and the temperature changing trend; and determine a heat balance temperature lower limit according to the real-time temperatures, the lower limit of the real-time temperature changing speed, and the temperature changing trend.

The detection result determination module 450 is configured to determine the temperature detection result of the detected person according to a relationship between the heat balance temperature higher limit and an alarm temperature threshold and a relationship between the heat balance temperature lower limit and the alarm temperature threshold.

The above-described product can execute the methods according to the embodiments of the present application, and have corresponding functional modules and effects for executing the methods.

A storage medium containing computer-executable instructions is further provided according to the embodiments of the present application, the computer-executable instructions are used to execute a temperature detection method when executed by a computer processor, the method includes the following.

Real-time temperatures of a detected person is acquired, a temperature changing trend of the detected person is determined to be a rising trend or a declining trend according to the real-time temperatures, and a real-time temperature changing speed is acquired according to the real-time temperatures.

A higher limit of the real-time temperature changing speed and a lower limit of the real-time temperature changing speed is determined according to the real-time temperature changing speed.

A temperature difference value corresponding to the higher limit of the real-time temperature changing speed is determined according to the higher limit of the real-time temperature changing speed and a temperature difference value corresponding to the lower limit of the real-time temperature changing speed is determined according to the lower limit of the real-time temperature changing speed.

A heat balance temperature higher limit is determined according to the real-time temperatures, the temperature difference value corresponding to the higher limit of the real-time temperature changing speed, and the temperature changing trend; and a heat balance temperature lower limit is determined according to the real-time temperatures, the lower limit of the real-time temperature changing speed, and the temperature changing trend.

The temperature detection result of the detected person is determined according to a relationship between the heat balance temperature higher limit and an alarm temperature threshold and a relationship between the heat balance temperature lower limit and the alarm temperature threshold.

Storage media are various types of memory devices or storage devices. The “storage medium” includes: an installation medium, such as a portable compact disc read only memory (CD-ROM), a floppy disk, or a tape apparatus; a computer system memory or random access memory, such as a dynamic random access memory (DRAM), a double data rate random access memory (DDR RAM), a static random access memory (SRAM), an extended data output random access memory (EDO RAM), a Rambus (RAM), etc.; a non-volatile memory, such as a flash memory, magnetic media (for example, a hard disk or optical storage); a register or other similar types of memory elements, etc. The storage medium may further include other types of memory or combinations thereof. In addition, the storage medium may be located in a computer system in which a program is executed, or may be located in a different second computer system connected to the computer system through a network such as the Internet. The second computer system may provide program instructions to the computer for execution. The term “storage medium” may include two or more storage media that may reside in different locations, such as in different computer systems connected by a network. A storage medium may store program instructions (e.g., implemented as a computer program) executable by one or more processors.

In a storage medium containing computer-executable instructions according to an embodiment of the present application, the computer-executable instructions are not limited to perform the above-described temperature detection operations, but can also perform related operations in the temperature detection method according to any embodiment of the present application.

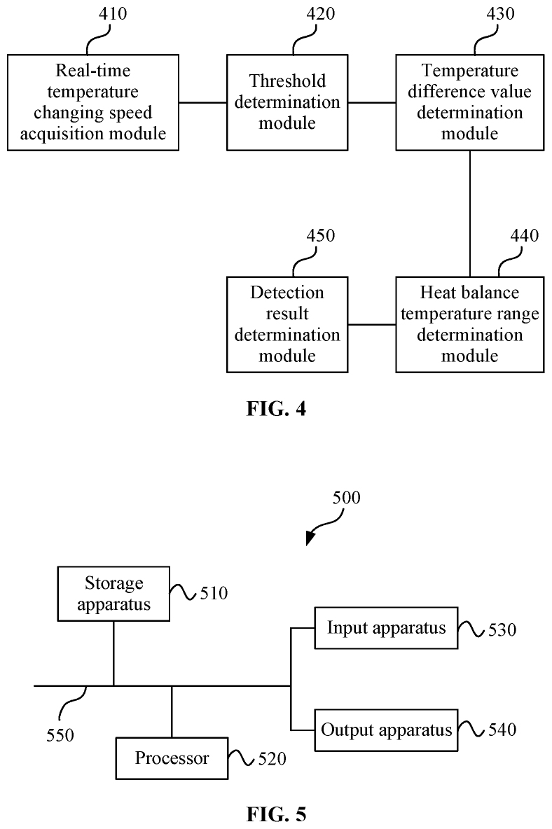

An electronic device is provided according to an embodiment of the present application, and the temperature detection apparatus according to the embodiment of the present application can be integrated into the electronic device. FIG. 5 is a schematic structural diagram of an electronic device according to an embodiment of the present application. As shown in FIG. 5, an electronic device 500 is provided according to this embodiment, which includes: one or more processors 520; a storage apparatus 510 for storing one or more programs, and the one or more programs, when being executed by the one or more processors 520, cause the one or more processors 520 to implement the temperature detection method according to the embodiment of the present application, the method includes the following.

Real-time temperatures of a detected person is acquired, a temperature changing trend of the detected person is determined to be a rising trend or a declining trend according to the real-time temperatures, and a real-time temperature changing speed is acquired according to the real-time temperatures.

A higher limit of the real-time temperature changing speed and a lower limit of the real-time temperature changing speed is determined according to the real-time temperature changing speed.

A temperature difference value corresponding to the higher limit of the real-time temperature changing speed is determined according to the higher limit of the real-time temperature changing speed and a temperature difference value corresponding to the lower limit of the real-time temperature changing speed is determined according to the lower limit of the real-time temperature changing speed.

A heat balance temperature higher limit is determined according to the real-time temperatures, the temperature difference value corresponding to the higher limit of the real-time temperature changing speed, and the temperature changing trend; and a heat balance temperature lower limit is determined according to the real-time temperatures, the lower limit of the real-time temperature changing speed, and the temperature changing trend.

The temperature detection result of the detected person is determined according to a relationship between the heat balance temperature higher limit and an alarm temperature threshold and a relationship between the heat balance temperature lower limit and the alarm temperature threshold.

The processor 520 further implements the technical solution of the temperature detection method according to any embodiment of the present application.

The electronic device 500 shown in FIG. 5 is only an example, and should not limit the functions and scope of use of the embodiments of the present application.

As shown in FIG. 5, the electronic device 500 includes a processor 520, a storage apparatus 510, an input apparatus 530, and an output apparatus 540; the number of processors 520 in the electronic device may be one or more, and in FIG. 5, one processor 520 is taken as an example; the processor 520, the storage apparatus 510, the input apparatus 530 and the output apparatus 540 in the electronic device may be connected through a bus or in other ways. In FIG. 5, the connection through the bus 550 is taken as an example.

The storage apparatus 510, as a computer-readable storage medium, may be used to store software programs, computer-executable programs and module units, such as program instructions corresponding to the temperature detection method in the embodiment of the present application.

The storage apparatus 510 may mainly include a program storage region and a data storage region, where the program storage region may store an operating system and an application program required by at least one function; the data storage region may store data created according to the use of the terminal, and the like. In addition, the storage apparatus 510 may include a high-speed random access memory, and may further include a non-volatile memory, for example, at least one magnetic disk storage device, a flash memory device, or other non-volatile solid-state storage devices. In some examples, the storage apparatus 510 may include a memory arranged remotely from the processor 520, and these remote memories may be connected through a network. Examples of the aforementioned networks include, but are not limited to, the internet, intranets, local area networks, mobile communication networks, and combinations thereof.

The input apparatus 530 may be used to receive input numbers, character information or voice information, and generate key signal inputs related to user settings and function control of the device. The output apparatus 540 may include devices such as a display screen and a speaker.

The electronic device according to an embodiment of the present application may calculate the heat balance temperature value by performing temperature trend detection, and determine the detection result of the human body temperature according to the relationship between the heat balance temperature value and the alarm temperature threshold, so as to achieve the object of improving the detection accuracy of the human body temperature.

The temperature detection apparatus, medium, and electronic device according to the above embodiments may execute the temperature detection method provided in any embodiment of the present application, and have corresponding functional modules for executing the method and achieve corresponding effects which can be achieved through executing the method. For technical details not exhaustively described in the foregoing embodiments, reference may be made to the temperature detection method provided in any embodiment of the present application.

1. A temperature detection method, comprising:
acquiring real-time temperatures of a detected person, determining a temperature changing trend of the detected person to be a rising trend or a declining trend according to the real-time temperatures, and acquiring a real-time temperature changing speed according to the real-time temperatures;
determining a higher limit of the real-time temperature changing speed and a lower limit of the real-time temperature changing speed according to the real-time temperature changing speed;
determining a temperature difference value corresponding to the higher limit of the real-time temperature changing speed according to the higher limit of the real-time temperature changing speed, and determining a temperature difference value corresponding to the lower limit of the real-time temperature changing speed according to the lower limit of the real-time temperature changing speed;
determining a heat balance temperature higher limit according to the real-time temperatures, the temperature difference value corresponding to the higher limit of the real-time temperature changing speed, and the temperature changing trend, and determining a heat balance temperature lower limit according to the real-time temperatures, the lower limit of the real-time temperature changing speed, and the temperature changing trend; and
determining a temperature detection result of the detected person according to a relationship between the heat balance temperature higher limit and an alarm temperature threshold and a relationship between the heat balance temperature lower limit and the alarm temperature threshold.

2. The method according to claim 1, wherein, the determining a temperature detection result of the detected person according to a relationship between the heat balance temperature higher limit and an alarm temperature threshold and a relationship between the heat balance temperature lower limit and the alarm temperature threshold comprises:
in a case where the temperature changing trend is the rising trend and the alarm temperature threshold is less than the heat balance temperature lower limit, determining that the temperature detection result is an abnormal high temperature; and in a case where the temperature changing trend is the rising trend and the alarm temperature threshold is greater than the heat balance temperature lower limit, determining that the temperature detection result is a normal body temperature; and
in a case where the temperature changing trend is the declining trend and the alarm temperature threshold is less than the heat balance temperature lower limit, determining that the temperature detection result is an abnormal high temperature; and in a case where the temperature changing trend is the declining trend and the alarm temperature threshold is greater than the heat balance temperature higher limit, determining that the temperature detection result is a normal body temperature.

3. The method according to claim 1, wherein,
before the determining a temperature detection result of the detected person according to a relationship between the heat balance temperature higher limit and an alarm temperature threshold and a relationship between the heat balance temperature lower limit and the alarm temperature threshold, the method further comprises: determining a balancing time higher limit according to the higher limit of the real-time temperature changing speed, and determining a balancing time lower limit according to the lower limit of the real-time temperature changing speed;
the determining a temperature detection result of the detected person according to a relationship between the heat balance temperature higher limit and an alarm temperature threshold and a relationship between the heat balance temperature lower limit and the alarm temperature threshold comprises:
in a case where the temperature changing trend is the rising trend and the alarm temperature threshold is greater than the heat balance temperature lower limit and less than the heat balance temperature higher limit, determining a relationship between an acceptable measurement time threshold and a balancing time for a latest one of the real-time temperatures to rise to the alarm temperature threshold; in a case where the balancing time for the latest one of the real-time temperatures to rise to the alarm temperature threshold is less than or equal to the acceptable measurement time threshold, continuing temperature measurement; and in a case where the balancing time for the latest one of the real-time temperatures to rise to the alarm temperature threshold is greater than the acceptable measurement time threshold, issuing a warning message of temperature instability; and
in a case where the temperature changing trend is the declining trend and the alarm temperature threshold is greater than the heat balance temperature lower limit and less than the heat balance temperature higher limit, determining a relationship between the acceptable measurement time threshold and a balancing time for the latest one of the real-time temperatures to decline to the alarm temperature threshold; in a case where the balancing time for the latest one of the real-time temperatures to decline to the alarm temperature threshold is less than or equal to the acceptable measurement time threshold, continuing temperature measurement; and in a case where the balancing time for the latest one of the real-time temperatures to decline to the alarm temperature threshold is greater than the acceptable measurement time threshold, issuing a warning message of temperature instability.

4. The method according to claim 1, wherein the determining a heat balance temperature higher limit according to the real-time temperatures, the temperature difference value corresponding to the higher limit of the real-time temperature changing speed, and the temperature changing trend, and determining a heat balance temperature lower limit according to the real-time temperatures, the lower limit of the real-time temperature changing speed, and the temperature changing trend comprises:
in a case where the temperature changing trend is the rising trend, determining that the heat balance temperature higher limit is the sum of a latest one of the real-time temperatures and the temperature difference value corresponding to the higher limit of the real-time temperature changing speed and the heat balance temperature lower limit is the sum of the latest one of the real-time temperatures and the temperature difference value corresponding to the lower limit of the real-time temperature changing speed; and
in a case where the temperature changing trend is the declining trend, determining that the heat balance temperature higher limit is a difference between the latest one of the real-time temperatures and the temperature difference value corresponding to the lower limit of the real-time temperature changing speed and the heat balance temperature lower limit is a difference between the latest one of the real-time temperatures and the temperature difference value corresponding to the higher limit of the real-time temperature changing speed.

5. The method according to claim 1, wherein
the real-time temperature changing speed comprise a temperature increasing speed and a temperature decreasing speed; and
the determining a higher limit of the real-time temperature changing speed and a lower limit of the real-time temperature changing speed according to the real-time temperature changing speed comprises: determining the higher limit of the real-time temperature changing speed according to the products of a preset higher limit coefficient and the temperature increasing speed, and determining the lower limit of the real-time temperature changing speed according to the products of a preset lower limit coefficient and the temperature decreasing speed.

6. The method according to claim 1, wherein the acquiring real-time temperatures of a detected person and determining a temperature changing trend of the detected person to be a rising trend or a declining trend according to the real-time temperatures, comprises:
sampling the real-time temperatures of the detected person according to a preset period;
determining an average slope of real-time temperature sampled points of the detected person through a preset temperature trend detection window; and
in a case where the average slope is greater than 0, determining that the temperature changing trend is the rising trend; and in a case where the average slope is less than 0, determining that the temperature changing trend is the declining trend.

7. The method according to claim 6, further comprising:
in a case where the temperature changing trend is the rising trend, calculating a temperature increasing speed in the preset temperature trend detection window and using the temperature increasing speed as the real-time temperature changing speed; and
in a case where the temperature changing trend is the declining trend, calculating a temperature decreasing speed in the preset temperature trend detection window and using the temperature decreasing speed as the real-time temperature changing speed.

8. (canceled)

9. A non-transitory computer-readable storage medium storing a computer program, wherein the processor, when executing the computer program, implements:
acquiring real-time temperatures of a detected person, determining a temperature changing trend of the detected person to be a rising trend or a declining trend according to the real-time temperatures, and acquiring a real-time temperature changing speed according to the real-time temperatures;
determining a higher limit of the real-time temperature changing speed and a lower limit of the real-time temperature changing speed according to the real-time temperature changing speed;
determining a temperature difference value corresponding to the higher limit of the real-time temperature changing speed according to the higher limit of the real-time temperature changing speed, and determining a temperature difference value corresponding to the lower limit of the real-time temperature changing speed according to the lower limit of the real-time temperature changing speed;
determining a heat balance temperature higher limit according to the real-time temperatures, the temperature difference value corresponding to the higher limit of the real-time temperature changing speed, and the temperature changing trend, and determining a heat balance temperature lower limit according to the real-time temperatures, the lower limit of the real-time temperature changing speed, and the temperature changing trend; and
determining a temperature detection result of the detected person according to a relationship between the heat balance temperature higher limit and an alarm temperature threshold and a relationship between the heat balance temperature lower limit and the alarm temperature threshold.

10. An electronic device, comprising a memory, a processor, and a computer program stored on the memory and operable on the processor, wherein, the processor, when executing the computer program, implements:
acquiring real-time temperatures of a detected person, determining a temperature changing trend of the detected person to be a rising trend or a declining trend according to the real-time temperatures, and acquiring a real-time temperature changing speed according to the real-time temperatures;
determining a higher limit of the real-time temperature changing speed and a lower limit of the real-time temperature changing speed according to the real-time temperature changing speed;
determining a temperature difference value corresponding to the higher limit of the real-time temperature changing speed according to the higher limit of the real-time temperature changing speed, and determining a temperature difference value corresponding to the lower limit of the real-time temperature changing speed according to the lower limit of the real-time temperature changing speed;
determining a heat balance temperature higher limit according to the real-time temperatures, the temperature difference value corresponding to the higher limit of the real-time temperature changing speed, and the temperature changing trend, and determining a heat balance temperature lower limit according to the real-time temperatures, the lower limit of the real-time temperature changing speed, and the temperature changing trend; and
determining a temperature detection result of the detected person according to a relationship between the heat balance temperature higher limit and an alarm temperature threshold and a relationship between the heat balance temperature lower limit and the alarm temperature threshold.

11. The non-transitory computer-readable storage medium according to claim 9, wherein the processor determines a temperature detection result of the detected person according to a relationship between the heat balance temperature higher limit and an alarm temperature threshold and a relationship between the heat balance temperature lower limit and the alarm temperature threshold by:
in a case where the temperature changing trend is the rising trend and the alarm temperature threshold is less than the heat balance temperature lower limit, determining that the temperature detection result is an abnormal high temperature; and in a case where the temperature changing trend is the rising trend and the alarm temperature threshold is greater than the heat balance temperature lower limit, determining that the temperature detection result is a normal body temperature; and
in a case where the temperature changing trend is the declining trend and the alarm temperature threshold is less than the heat balance temperature lower limit, determining that the temperature detection result is an abnormal high temperature; and in a case where the temperature changing trend is the declining trend and the alarm temperature threshold is greater than the heat balance temperature higher limit, determining that the temperature detection result is a normal body temperature.

12. The non-transitory computer-readable storage medium according to claim 9, wherein,
before the processor determines the temperature detection result of the detected person according to the relationship between the heat balance temperature higher limit and the alarm temperature threshold and the relationship between the heat balance temperature lower limit and the alarm temperature threshold, the processor is further configured to determine a balancing time higher limit according to the higher limit of the real-time temperature changing speed, and determining a balancing time lower limit according to the lower limit of the real-time temperature changing speed;
the processor determines the temperature detection result of the detected person according to the relationship between the heat balance temperature higher limit and the alarm temperature threshold and the relationship between the heat balance temperature lower limit and the alarm temperature threshold by:
in a case where the temperature changing trend is the rising trend and the alarm temperature threshold is greater than the heat balance temperature lower limit and less than the heat balance temperature higher limit, determining a relationship between an acceptable measurement time threshold and a balancing time for a latest one of the real-time temperatures to rise to the alarm temperature threshold; in a case where the balancing time for the latest one of the real-time temperatures to rise to the alarm temperature threshold is less than or equal to the acceptable measurement time threshold, continuing temperature measurement; and in a case where the balancing time for the latest one of the real-time temperatures to rise to the alarm temperature threshold is greater than the acceptable measurement time threshold, issuing a warning message of temperature instability; and
in a case where the temperature changing trend is the declining trend and the alarm temperature threshold is greater than the heat balance temperature lower limit and less than the heat balance temperature higher limit, determining a relationship between the acceptable measurement time threshold and a balancing time for the latest one of the real-time temperatures to decline to the alarm temperature threshold; in a case where the balancing time for the latest one of the real-time temperatures to decline to the alarm temperature threshold is less than or equal to the acceptable measurement time threshold, continuing temperature measurement; and in a case where the balancing time for the latest one of the real-time temperatures to decline to the alarm temperature threshold is greater than the acceptable measurement time threshold, issuing a warning message of temperature instability.

13. The non-transitory computer-readable storage medium according to claim 9, wherein the processor determines the heat balance temperature higher limit according to the real-time temperatures, the temperature difference value corresponding to the higher limit of the real-time temperature changing speed, and the temperature changing trend, and determines the heat balance temperature lower limit according to the real-time temperatures, the lower limit of the real-time temperature changing speed, and the temperature changing trend by:
in a case where the temperature changing trend is the rising trend, determining that the heat balance temperature higher limit is the sum of a latest one of the real-time temperatures and the temperature difference value corresponding to the higher limit of the real-time temperature changing speed and the heat balance temperature lower limit is the sum of the latest one of the real-time temperatures and the temperature difference value corresponding to the lower limit of the real-time temperature changing speed; and
in a case where the temperature changing trend is the declining trend, determining that the heat balance temperature higher limit is a difference between the latest one of the real-time temperatures and the temperature difference value corresponding to the lower limit of the real-time temperature changing speed and the heat balance temperature lower limit is a difference between the latest one of the real-time temperatures and the temperature difference value corresponding to the higher limit of the real-time temperature changing speed.

14. The non-transitory computer-readable storage medium according to claim 9, wherein
the real-time temperature changing speed comprise a temperature increasing speed and a temperature decreasing speed; and
the processor determines the higher limit of the real-time temperature changing speed and the lower limit of the real-time temperature changing speed according to the real-time temperature changing speed by: determining the higher limit of the real-time temperature changing speed according to the products of a preset higher limit coefficient and the temperature increasing speed, and determining the lower limit of the real-time temperature changing speed according to the products of a preset lower limit coefficient and the temperature decreasing speed.

15. The non-transitory computer-readable storage medium according to claim 1, wherein the processor acquires the real-time temperatures of the detected person and determines the temperature changing trend of the detected person to be the rising trend or the declining trend according to the real-time temperatures by:
sampling the real-time temperatures of the detected person according to a preset period;
determining an average slope of real-time temperature sampled points of the detected person through a preset temperature trend detection window; and
in a case where the average slope is greater than 0, determining that the temperature changing trend is the rising trend; and in a case where the average slope is less than 0, determining that the temperature changing trend is the declining trend.

16. The non-transitory computer-readable storage medium of claim 15, where the processor is further configured to implement:
in a case where the temperature changing trend is the rising trend, calculating a temperature increasing speed in the preset temperature trend detection window and using the temperature increasing speed as the real-time temperature changing speed; and
in a case where the temperature changing trend is the declining trend, calculating a temperature decreasing speed in the preset temperature trend detection window and using the temperature decreasing speed as the real-time temperature changing speed.

17. The electronic device according to claim 10, wherein the processor determines a temperature detection result of the detected person according to a relationship between the heat balance temperature higher limit and an alarm temperature threshold and a relationship between the heat balance temperature lower limit and the alarm temperature threshold by:
in a case where the temperature changing trend is the rising trend and the alarm temperature threshold is less than the heat balance temperature lower limit, determining that the temperature detection result is an abnormal high temperature; and in a case where the temperature changing trend is the rising trend and the alarm temperature threshold is greater than the heat balance temperature lower limit, determining that the temperature detection result is a normal body temperature; and
in a case where the temperature changing trend is the declining trend and the alarm temperature threshold is less than the heat balance temperature lower limit, determining that the temperature detection result is an abnormal high temperature; and in a case where the temperature changing trend is the declining trend and the alarm temperature threshold is greater than the heat balance temperature higher limit, determining that the temperature detection result is a normal body temperature.

18. The electronic device according to claim 10, wherein,
before the processor determines the temperature detection result of the detected person according to the relationship between the heat balance temperature higher limit and the alarm temperature threshold and the relationship between the heat balance temperature lower limit and the alarm temperature threshold, the processor is further configured to determine a balancing time higher limit according to the higher limit of the real-time temperature changing speed, and determining a balancing time lower limit according to the lower limit of the real-time temperature changing speed;
the processor determines the temperature detection result of the detected person according to the relationship between the heat balance temperature higher limit and the alarm temperature threshold and the relationship between the heat balance temperature lower limit and the alarm temperature threshold by:
in a case where the temperature changing trend is the rising trend and the alarm temperature threshold is greater than the heat balance temperature lower limit and less than the heat balance temperature higher limit, determining a relationship between an acceptable measurement time threshold and a balancing time for a latest one of the real-time temperatures to rise to the alarm temperature threshold; in a case where the balancing time for the latest one of the real-time temperatures to rise to the alarm temperature threshold is less than or equal to the acceptable measurement time threshold, continuing temperature measurement; and in a case where the balancing time for the latest one of the real-time temperatures to rise to the alarm temperature threshold is greater than the acceptable measurement time threshold, issuing a warning message of temperature instability; and
in a case where the temperature changing trend is the declining trend and the alarm temperature threshold is greater than the heat balance temperature lower limit and less than the heat balance temperature higher limit, determining a relationship between the acceptable measurement time threshold and a balancing time for the latest one of the real-time temperatures to decline to the alarm temperature threshold; in a case where the balancing time for the latest one of the real-time temperatures to decline to the alarm temperature threshold is less than or equal to the acceptable measurement time threshold, continuing temperature measurement; and in a case where the balancing time for the latest one of the real-time temperatures to decline to the alarm temperature threshold is greater than the acceptable measurement time threshold, issuing a warning message of temperature instability.

19. The electronic device according to claim 10, wherein the processor determines the heat balance temperature higher limit according to the real-time temperatures, the temperature difference value corresponding to the higher limit of the real-time temperature changing speed, and the temperature changing trend, and determines the heat balance temperature lower limit according to the real-time temperatures, the lower limit of the real-time temperature changing speed, and the temperature changing trend by:
in a case where the temperature changing trend is the rising trend, determining that the heat balance temperature higher limit is the sum of a latest one of the real-time temperatures and the temperature difference value corresponding to the higher limit of the real-time temperature changing speed and the heat balance temperature lower limit is the sum of the latest one of the real-time temperatures and the temperature difference value corresponding to the lower limit of the real-time temperature changing speed; and
in a case where the temperature changing trend is the declining trend, determining that the heat balance temperature higher limit is a difference between the latest one of the real-time temperatures and the temperature difference value corresponding to the lower limit of the real-time temperature changing speed and the heat balance temperature lower limit is a difference between the latest one of the real-time temperatures and the temperature difference value corresponding to the higher limit of the real-time temperature changing speed.

20. The electronic device according to claim 10, wherein
the real-time temperature changing speed comprise a temperature increasing speed and a temperature decreasing speed; and
the processor determines the higher limit of the real-time temperature changing speed and the lower limit of the real-time temperature changing speed according to the real-time temperature changing speed by: determining the higher limit of the real-time temperature changing speed according to the products of a preset higher limit coefficient and the temperature increasing speed, and determining the lower limit of the real-time temperature changing speed according to the products of a preset lower limit coefficient and the temperature decreasing speed.
